(19) United States
(12) Patent Application Publication
(10) Pub. No.: US 2013/0022669 A1
(43) Pub. Date: Jan. 24, 2013

(54) BRANCHED, COMPACT POLYETHYLENEGLYCOL DERIVATIVES

(71) Applicants: Oskar Axelsson, Hoor (SE); Fredrik Ek, Lund (SE)

(72) Inventors: Oskar Axelsson, Hoor (SE); Fredrik Ek, Lund (SE)

(73) Assignee: SPAGO IMAGING AB, Stockholm (SE)

(21) Appl. No.: 13/636,771

(22) PCT Filed: Mar. 29, 2011
(86) PCT No.: PCT/SE11/50346

§ 371 Date: Oct. 8, 2012

(30) Foreign Application Priority Data
Mar. 30, 2010 (SE) 1050301-9

(51) Int. Cl. C07C 43/11; C07K 16/00; C07H 21/02; C07H 15/00; A61K 9/16; A61K 9/127; C07C 69/66; A61M 25/00; A61N 1/05; C07K 14/00; C07D 493/18; B82Y 5/00

(52) U.S. Cl. 424/450; 568/679; 530/300; 530/387.1; 536/23.1; 536/4.1; 549/354; 424/490; 560/179; 604/264; 607/116; 977/773; 977/906

(57) ABSTRACT

The present invention relates to branched polyethyleneglycol (PEG) derivatives consisting of a molecule with a quaternary carbon connected to three PEG chains, wherein all three PEG chains are of equal length and each comprises 1-30 —OCH₂CH₂— units, and one group with at least one carbon atom, wherein said at least one carbon atom is attached to the quaternary carbon. Also compositions containing such derivatives and use of such derivatives are disclosed.

### FIELD OF THE INVENTION

The present invention relates to branched polyethyleneglycol (PEG) derivatives with advantages over currently available PEG derivatives. They are especially applicable for conjugation with nanoparticles, proteins, peptides, pharmaceutically active small molecules, liposomes, homogenous catalysts and bio-inert surfaces.

### BACKGROUND

Bio-inert polar polymers, and in particular polyethyleneglycol (HO(CH₂CH₂O)H, PEG, also called polyethyleneoxide; PEO), have found extensive technical use, notably in the area of pharmaceuticals and medical devices. Lately covalent grafting with PEG have been used to modify proteins, peptides, oligonucleotides, antibody fragments, small molecule drugs, catalysts, surfaces and particles. These modified conjugates often obtain superior properties such as decreased immunogenicity or antigenicity, improved pharmacokinetic and pharmadynamic properties, enhanced solubility in aqueous media, increased stability and reduced clearance rate from the body.

Several PEG-derivatives of macromolecules have now been approved for pharmaceutical use. The first one was PEG-adenosine deaminase for treatment of adenosine deaminase deficiency (ADA deficiency).

Organic catalysts have also been covalently modified with PEG-derivatives to allow reactions in water (Hong and Grubbs, J. Am. Chem. Soc. 2006, 128, 3508).

Traditionally, polydisperse linear PEG-derivatives have been used for PEG grafting but recently monodisperse PEG derivatives or PEG derivatives of defined mass have been applied to obtain homogeneous products, simplifying characterization of the PEG-derivatives, thus facilitating the regulatory process.

Branched PEG, characterized by two or more PEG chains linked to a common center, has been developed as an alternative to the more common linear PEG derivatives. Monfardini et al. (Bioconj. Chem. 1995, 6, 62-69) reported that surface modification of proteins using branched PEG derivatives resulted in increased biocompatibility and better stability. These PEGs allows for a better masking and protection of the connected surface/molecule. In addition, the modification of protein using branched PEG provides a more efficient usage of amino acids/amino acid side chains available for chemical linking, thus more PEG polymer per modification. Fewer chemical modifications of the protein increase the probability of retaining the native structure of the biomolecule and hence the biological activity.

The strategies for attaching PEG to macromolecules and small organic molecules, respectively, differ in terms of size and number of PEG molecules that can be attached. Large macromolecules are commonly modified using PEG polymer of molecular weights ranging from 5 kDa to 90 kDa to obtain reduced clearance from the blood, better specificity while preserving biological activity. However, large PEG molecules are not suitable for modifications of small organic molecules. Conjugates of pharmaceutically active small molecules and large PEG polymers prevents, in most cases, the conjugate to interact with receptors or binding pockets due to sterical hindrance or even alter the ability of the molecule to diffuse through membranes to reach their target. Conjugates with large PEG moieties have low diffusion rates, just like any large molecules. Therefore, PEG derivatives with molecular weights of less than 2 kDa are generally used for modification of small organic molecules. (Ouchi, T. in Poly(ethylene glycol): Chemistry and biological applications, Harris and Zalipsky Eds, Chapter 19). In general, macromolecules tolerate attachment of PEG to several positions using smaller PEG although it is normally beneficial to have as few as possible. On the other hand, small organic compounds have few points for the attachment of PEG and it is much more difficult to obtain conjugates that retain the unmodified molecule's bioactive properties.

In WO 95/025763 is disclosed branched PEG-like structures (as part of a larger structure) which have a superficial likeness to the structures present invention but lack the essential feature of being discrete and useful for the applications disclosed here.

In WO 2007/025763 is disclosed branched PEG derivatives of defined character. However, their structure differs from the present invention in the following non-beneficial ways; They utilize 2,2,2-tris(hydroxymethyl)methylamine as the core structure so the detailed structures are more complex and thus more expensive to produce. The core is based on amides and hence more sensitive to hydrolysis, and the individual branched PEG derivatives contain thioethers, a functionality known for being notoriously sensitive to oxidization, which can be a further source of heterogeneity of the product. The core structure is not compact and thus not useful for some of the applications disclosed here.

### DEFINITIONS OF TERMS

The term “nanoparticle” is used to describe a particle of any shape with a longest measure from 1-100 nm.

“Bio-inert” refers to a material that is bio-compatible, i.e. harmless to a living organism and at the same time stable to degradation in-vivo.

“Monolayer” refers to a one molecule thick layer.

“Oriented” in the context of coatings refers to a layer of coating molecules where all the heads and tails (as arbitrarily defined from case to case but as intended in the present invention we consistently refer to the silane, where present, as the head) of the coating molecules are oriented in the same way in relation to the particle core surface.

“Activated silane” refers to a silane of the following type RₙSi(X)₄₋ₙ,

where X is an alkoxy group, aryloxy group, a halogen, a dialkylamino group, a nitrogen containing heterocycle or an acyloxy group and R is an organic group.

“Oxysilane” refers to any organic compounds with one or more oxygen atoms attached to the silicon atom. Non-limiting examples thereof are:

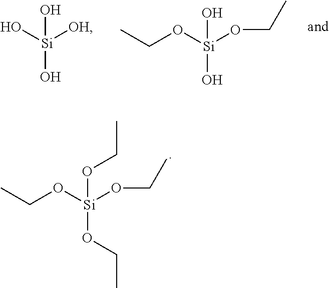

“Organosilane” refers to organic compounds containing one or more carbon silicon bonds.

“Organic residue” refers to organic compounds covalently bond to a molecular entity.

“Organo-oxysilane” refers to organic compounds containing one or more carbon atoms and one or more oxygen atoms attached to the silicon atom. Non-limiting examples thereof are:

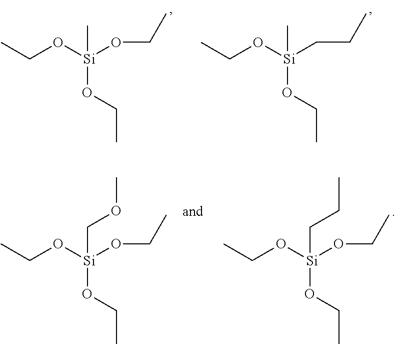

“Hydrocarbon” or “hydrocarbon chain” is an organic residue consisting of hydrogen and carbon. As used in the present invention a hydrocarbon may, when indicated, comprise heteroatoms selected from O, S and N. This means that one or more of the carbon atoms have been replaced by a heteroatom selected from O, S or N. The hydrocarbon may be fully saturated or it may comprise one or more unsaturations. Unless otherwise specified, the hydrocarbon may contain any number of carbon atoms between 1 and 50. The hydrocarbon group of the compounds may then be designated as “C₁₋₈ hydrocarbon” or similar designations. Typical hydrocarbon groups include, but are in no way limited to, methyl, ethyl, propyl, isopropyl, butyl, isobutyl, tertiary butyl, pentyl, hexyl, ethenyl, propenyl, butenyl, phenyl, benzyl.

“Alkyl” refers to a straight or branched hydrocarbon chain fully saturated (no double or triple bonds) hydrocarbon group. The alkyl group may have 1 to 8 carbon atoms. The alkyl group of the compounds may be designated as “C₁₋₈ alkyl” or similar designations. Typical alkyl groups include, but are in no way limited to, methyl, ethyl, propyl, isopropyl, butyl, isobutyl, tertiary butyl, pentyl, hexyl, and the like.

Whenever it appears herein, a numerical range such as “from 1 to 8” or “1-8” refer to each integer in the given range; e.g., “from 1 to 8 carbon atoms” means that the alkyl group may consist of 1 carbon atom, 2 carbon atoms, 3 carbon atoms, etc., up to and including 8 carbon atoms.

As used herein, “alkoxy” refers to the formula —OR wherein R is a C₁₋₈ alkyl, e.g. methoxy, ethoxy, n-propoxy, 1-methylethoxy (isopropoxy), nbutoxy, iso-butoxy, sec-butoxy, tert-butoxy, amyloxy, iso-amyloxy and the like. An alkoxy may be optionally substituted.

As used herein, “aryloxy” refers to RO— in which R is an aryl wherein, “aryl” refers to a carbocyclic (all carbon) ring or two or more fused rings (rings that share two adjacent carbon atoms) that have a fully delocalized pielectron system. Examples of aryl groups include, but are not limited to, benzene, naphthalene and azulene. An aryl group may be optionally substituted, e.g., phenoxy, naphthalenyloxy, azulenyloxy, anthracenyloxy, naphthalenylthio, phenylthio and the like. An aryloxy may be optionally substituted As used herein, “acyl” refers to a carbonyl group, i.e. —C(═O)—.

As used herein, “acyloxy” refers to an oxygen atom connected via a carbonyl group, i.e. —C(═O)—O—.

As used herein, “heterocycle” refers to a stable 3- to 18 membered ring which consists of carbon atoms and from one to five heteroatoms selected from the group consisting of nitrogen, oxygen and sulfur. The heterocycle may be monocyclic, bicyclic or tricyclic.

“Strong base” refers in the current context to bases that are stronger than hydroxide and not compatible with aqueous environments.

“Conjugate” refers to a molecular entity that is a fluorescence marker, dye, spin-label, radioactive marker, ligand to a biological receptor, chelate, enzyme inhibitor, enzyme substrate, antibody or anti-body related structure. See e.g. “Bioconjugate Techniques”, Greg T. Hermanson second edition, Elsevier 2008, ISBN 978-0-12-370501-3 for background on the subject.

“Handle for conjugation” or “attachment point” refers to a bifunctional molecule that can bind to, or be incorporated in, the silane coating but leaving one reactive group that can be linked to a conjugate, as defined above. A typical, but not exclusive, example would be (EtO)₃SiCH₂CH₂CH₂NH₂.

“m-PEG” refers to structures CH₃—(OCH₂CH₂)ₙ—OH where n depends on the circumstances.

“Aprotic solvent” refers to solvents which have no protons that can be removed or rapidly exchanged in an aqueous environment. Typical, but not limiting, examples of such solvents are tetrahydrofuran (THF), diethyl ether, glyme, diglyme, dimethyl formamide (DMF), dimethylsulfoxide, or N-methyl pyrrolidinone (NMP).

DCM is an acronym for dichloromethane.

### DETAILED DESCRIPTION OF THE INVENTION

A first aspect of the present invention is a branched polyethyleneglycol (PEG) derivative comprising three PEG chains attached to a common quaternary carbon atom. It may be preferred that all three PEG chains are of equal length and each comprises 1-30-OCH₂CH₂— units The fourth group attached to said quaternary carbon atom can be any of a large number of different organic groups. This forth group shall contain a carbon atom, which is attached to the quaternary carbon atom. A non-limiting list of examples of the fourth group attached to the quaternary carbon atom is given in the list describing group R¹ below.

The branched polyethyleneglycol (PEG) derivative according to the invention may have formula I or formula II:

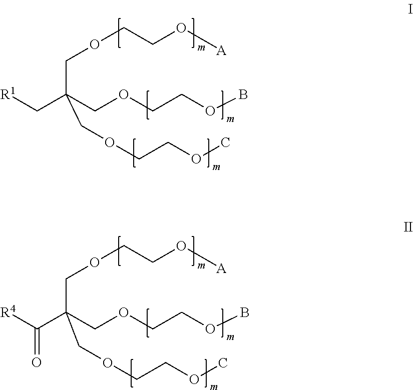

wherein:

m is selected from 1-30;

R¹ is selected from the list given in Table 1 below;

A, B and C are independently selected from —C₁-C₈ hydrocarbon, —(CH₂)₂N₃, —(CH₂)₂NR⁵₂, CH₂COOR⁴;

R² is selected from C₁-C₈ hydrocarbon;

R³ is selected from C₁₋₈ alkoxy group, aryloxy group, a halogen, a diC₁₋₈-alkylamino group, a nitrogen containing heterocycle or an acyloxy group;

R⁴ is selected from H, OH, OR², NHR², N(R²)₂, halogen, N-hydroxysuccinimidyl (NHS ester) and perfluorophenolate, wherein R² is as above;

R⁵ is independently selected from —H, —C₁-C₈ hydrocarbon, —(C═O)NH₂, —(C═O)—OCH₃, —(C═O)OCH₂CH₃.

TABLE 1
Different options for R¹

| Variant of formula I | R¹ |
|---|---|
| Ia | —OH |
| Ib | —OSO₂CH3 |
| Ic | —OSO₂PhCH3 |
| Id | —OCH₂CH═CH₂ |
| Ie | -Obenzyl |
| If | -halogen |
| Ig | —NH₂ |
| Ih | —NHR² |
| Ii | —N(R²)₂ |
| Ij | —NHCO₂R² |
| Ik | —NHCONHR² |
| Il | —NHCON(R²)₂ |
| Im | —NCO |
| In | —NCS |
| Io | —NHCO(CH₂)₄CH(SH)CH₂CH₂SH |
| Ip | —NHCOCH₂SH |
| Iq | —SH |
| Ir | —SR² |
| Is | —SO₃H |
| It | —SO₂Cl |
| Iu | —S═OR² |
| Iv | —SO₂R² |
| Ix | —SONHR² |
| Iy | —O(CH₂)₃SH |
| Iz | —OCH₂CH(SH)CH₂SH |
| Iaa | —OCO(CH₂)₄CH(SH)CH₂CH₂SH |
| Iab | —PO₃H₂ |
| Iac | —PO3(R²)₂ |
| Iad | —OCH₂COOH |
| Iae | —OCH₂COR⁴ |
| Iaf | —O(CH₂)₂NH₂ |
| Iag | —O(CH₂)₂NHR² |
| Iah | —O(CH₂)₂N(R²)₂ |
| Iai | —O(CH₂)₂NHCO₂R² |
| Iaj | —O(CH₂)₂NHCONHR² |
| Iak | —O(CH₂)₂NHCON(R²)₂ |
| Ial | —O(CH₂)₂NCO |
| Iam | —O(CH₂)₃Si(R³)₃ |
| Ian | —O(CH₂)₂NHCO(CH₂)₄CH(SH)CH₂CH₂SH |

In some embodiments R¹ in formula I is O(CH₂)₃Si(R³)₃, and R³ may then be selected from the group consisting of O₁₋₈ alkoxy, acyloxy, dialkylamino and aryloxy.

In some embodiments R¹ in formula I is CH₂NH₂ or OCH₂COOH.

In some embodiments m is 3-20.

In some embodiments m is 3-10.

In some embodiments m is 3-5.

In some embodiments A, B and C are all equal.

In some embodiments A, B and C are all —CH₃. Alternatively, the branched polyethyleneglycol (PEG) derivative according to the invention may have formula:

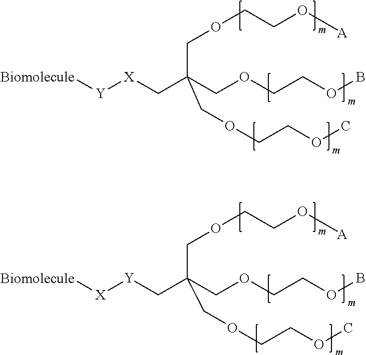

where “Biomolecule” denotes a peptide or an antibody fragment or a ribonucleic acid or a carbohydrate and X is a residue of the functionality used for coupling and Y is a spacer group with structure —(OCH2CH2)ₙO— where n is selected from 1-50 and m is selected from 2-30 and A, B and C are independently selected from —C₁-C₈ hydrocarbon, —(CH₂)₂N₃, —(CH₂)₂NR⁵₂ and —CH₂COOR⁴; and; R² is selected from C₁-C₈ hydrocarbon; and R³ is selected from C₁₋₈ alkoxy group, aryloxy group, a halogen, a di-C₁₋₈-alkylamino group, a nitrogen containing heterocycle or an acyloxy group and; R⁴ is selected from H, OH, OR², NHR², N(R²)₂, halogen, N-hydroxysuccinimidyl (NHS ester) and perfluorophenolate; and R⁵ is independently selected from —H, —C₁-C₈ hydrocarbon, —(C═O)NH₂, —(C═O)—OCH₃, or —OCH₂CH₃.

Alternatively, the compound according to the invention may have one of the two formulas:

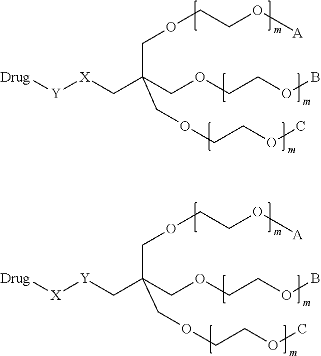

wherein “Drug” denotes a pharmaceutically active drug of molecular weight less than 1000 g/mol and X is a residue of the functionality used for coupling and Y is a spacer group with structure —(OCH2CH2)ₙO— where n is selected from 1-50 and m is selected from 2-30 and A, B and C are independently selected from —C₁-C₈ hydrocarbon, —(CH₂)₂N₃, —(CH₂)₂NR⁵₂ and —CH₂COOR⁴; and; R² is selected from C₁-C₈ hydrocarbon; and R³ is selected from C₁₋₈ alkoxy group, aryloxy group, a halogen, a di-C₁₋₈-alkylamino group, a nitrogen containing heterocycle or an acyloxy group and; R⁴ is selected from H, OH, OR², NHR², N(R²)₂, halogen, N-hydroxysuccinimidyl (NHS ester) and perfluorophenolate; and R⁵ is independently selected from —H, —C₁-C₈ hydrocarbon, —(C═O)NH₂, or —(C═O)—OCH₃.

Alternatively, the compound according to the invention may have formula Va

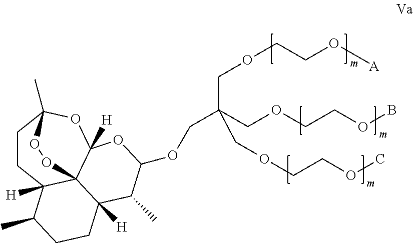

wherein m=1-30 and A, B, and C independently are selected from the group consisting of H and methyl.

Alternatively, the compound according to the invention may have formula IXa

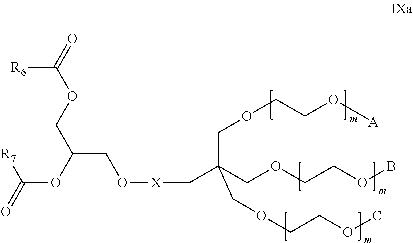

wherein:

R⁶ and R⁷ are independently selected from the group consisting of C₈-C₂₅ hydrocarbons;

m is selected from 2-30;

A, B and C are independently selected from the group consisting of —C₁-C₈ hydrocarbon, —(CH₂)₂N₃, —(CH₂)₂NR⁵₂ and —CH₂COOR⁴;

R² is selected from C₁-C₈ hydrocarbon;

R³ is selected from C₁₋₈ alkoxy group, aryloxy group, a halogen, a diC₁₋₈-alkylamino group, a nitrogen containing heterocycle or an acyloxy group;

R⁴ is selected from H, OH, OR², NHR², N(R²)₂, halogen, N-hydroxysuccinimidyl (NHS ester) and perfluorophenolate;

R⁵ is independently selected from —H, —C₁-C₈ hydrocarbon, —(C═O)NH₂, —(C═O)—OCH₃, or —OCH₂CH₃;

X is a bond, a chain or a ring structure with 0-10 linear atoms chosen independently from C, O, N, P and S to form chemical compounds according to normal valency rules; and

Crucial to some embodiments of the invention is that the composition is essentially free from molecules where any of the chain lengths m have a different value than the desired one, i.e. the product is of defined molecular weight or, synonymously, monodisperse; so that the purity of the product is more than 50%. Suitable purities are for example more than 80% more than 90%, more than 95%, more than 99%. In one embodiment of the present invention the purity is above 95%.

This type of PEG derivatives are readily produced from inexpensive starting materials using a concise synthetic strategy outlined in scheme 3. Compounds according to formula I also has the advantage of being economically viable. In particular are the starting materials for I with m=3 or 4 favourable. The following calculation shows the economical advantage of the present invention: If R₁ in structure I is —OH and m=4, the molecular weight of the product is 707 g/mol. The cost of the starting materials are 20 $/g product when bought on a small scale. Commercially available linear m-PEGS with a similar molecular weight are isolated from PEG mixtures using expensive and time consuming chromatography. A typical example is m-PEG with 11 repeating ethylene glycol units, which is available at a cost of $ 240/g (Polypure, Norway). A branched amide based monodisperse PEG analog is available from Quanta Biodesign at $1300/g.

The present invention thus enables formation of PEG derivatives based on low cost starting materials.

PEG alcohols of defined molecular weight are commercially available up to lengths of about 30 monomer units so the present invention relates to generic structures I wherein m is 1-30, or for example 2-20, 3-12; 3-6 and 3-4. In one embodiment m is 3-4.

Depending on the origin of the PEG alcohol used in the synthesis of the branched PEG derivatives of the present invention, the final product will have a spectrum of minor impurities. Some of those impurities may not have m in general structure I or II equal and are thus not a desired material but considered as impurities. In general, it is advisable to use pure starting materials. Suitable purities of the starting materials are higher than 70% or higher than 90% or higher than 95% or higher than 99%. There may also be residues from incomplete reaction of the intermediates which can be minimized on a case by case basis by optimizing reaction time, reaction temperature, amount of solvent, identity of the solvent, or identity of the base.

A second aspect of the present invention relates to the process of producing molecules according to the first aspect. Products I or II are possible to produce by several routes obvious to the one skilled in the art but the process that is described here as a second aspect of this invention has the advantage of using a cheap, commercially available trihaloalcohol as a key intermediate and source of the quaternary carbon as outlined in scheme 3.

The process comprises the steps of:
a. Contacting the key starting material 3-halo-2,2-bis(halomethyl)propanol, preferably in the form of 3-bromo-2,2-bis(bromomethyl)propanol, with an allylation reagent such as, allyl chloride, allyl mesylate, or allyl tosylate or, preferably, allyl bromide, optionally in a solvent inert to the reaction conditions, in the presence of a strong base, for example sodium hydride. The intermediate may then be isolated by any standard technique described in organic chemistry textbooks (Advanced practical organic chemistry, Leonard, Lygo and Procter 1998, 2^{nd} ed, Stanley Thornes Publishers, Cheltenham). It is also conceivable to proceed with the next step without isolating the intermediate 3-(3-bromo-2,2-bis(bromomethyl)propoxy)prop-1-ene.
b. Contacting the intermediate product with a PEG-oligomer of structure A-(OCH₂CH₂)ₘO—; or base and A-(OCH₂CH₂)ₘOH; where A is selected from —C₁-C₈ hydrocarbon, —(CH₂)₂N₃, —(CH₂)₂NR⁵₂ and —CH₂COOR⁴; and; R² is selected from C₁-C₈ hydrocarbon; and R³ is selected from C₁₋₈ alkoxy group, aryloxy group, a halogen, a di-C₁₋₈-alkylamino group, a nitrogen containing heterocycle or an acyloxy group and; R⁴ is selected from H, OH, OR², NHR², N(R²)₂, halogen, N-hydroxysuccinimidyl (NHS ester) and perfluorophenolate; and R⁵ is independently selected from —H, —C₁-C₈ hydrocarbon, (C═O)NH₂, —(C═O)—OCH₃, —OCH₂CH₃ and m is selected from 1-30, with a defined molecular weight, optionally in a solvent inert to the reaction conditions, in the presence of a strong base, such as sodium hydride. The temperature in this step may advantageously be above room temperature such as between 30 and 150° C. or between 70 and 120° C. or preferably between 90 and 110° C. Optionally the resulting branched PEG intermediate may be isolated by any standard technique described in organic chemistry textbooks (Advanced practical organic chemistry, Leonard, Lygo and Procter 1998, 2^{nd} ed, Stanley Thornes Publishers, Cheltenham).
c. The intermediate of step b is then transformed into the actual derivatizing agent by any of the many methods obvious to one skilled in the art and some but not limiting examples are discussed below and further elaborated in examples 3-6.
In one embodiment an extractive procedure or vacuum distillation was found to be favourable to use in a).

In one embodiment an extractive procedure followed by a simple chromatography was found to be favourable to use in b).

If the allyl group of Id is removed by a standard protective group manipulation, e.g. DMSO/KOt-Bu (Potassium tert-butoxide), followed by HCl (hydrochloric acid) (example 4) the ensuing alcohol Ia can be elaborated into several other functional groups. Some, non limiting, examples are given here: mild oxidation of the hydroxyl group of Ia with e.g. pyridinium chloro chromate or Dess-Martin periodinane gives the aldehyde II (R⁴═H) whereas stronger oxidizing conditions like fuming nitric acid or potassium permanganate give the corresponding carboxylic acid II (R⁴═OH). The carboxylic acid can in turn be activated and derivatized in a multitude of ways. If the aldehyde is subjected to reductive amination conditions i.e. NaCNBH₃/NH₃ the amine Ig is obtained. If the alcohol instead it treated with a halogenation reagent such as SOCl₂ or PBr₃, the halogen compound Iu is obtained. This can in turn be used to produce the thiol Iq, for example, by reaction with thiourea followed by hydrolysis in NaOH/EtOH. The thiol can in turn be alkylated to Ir or oxidized to the sulfonic acid Is. It can then be oxidized to the sulfoxide Iu or the sulfone Iv. Alternatively can lu be converted to the phosphonate Iac by reaction with dimethyl or diethyl phosphite. This can be hydrolyzed to the corresponding phosphonic acid Iab by treatment with trimethyl silylbromide.

To obtain Iam with Z═(R³)₃Si(CH₂)₃O—, where R³ is as defined in table 1, from the intermediate Id, the method of hydrosilylation is preferred. This involves adding a trialkoxy silane across the double bond in the presence of a catalyst. Preferred are catalysts based on platinum and, in particular, Karstedt's catalyst has been found to be effective.

Directly accessible from Id is the aldehyde Iae (R⁴═H) by means of two alternative procedures. On a small scale ozonization followed by a reductive work up can be contemplated. On a larger scale, reaction with osmium tetroxide to give the vicinal dialcohol followed by cleavage with periodic acid, is preferred.

Further advantages of the above process are that it is short and that the branched PEG-derivative is free from labile bonds. The commercially available branched PEG derivatives are based on amides and hence more sensitive to hydrolysis, which can be a further source of heterogeneity of the product and hence lead to regulatory complications during development for clinical use.

In a third aspect of the present invention, a composition comprising the general structures I or II covalently linked to a nanosized material, such as a nanoparticle, to enhance its properties with regard to solubility, viscosity, stability, biological compatibility, or pharmacokinetics or pharmacodynamics, is contemplated. For the purpose of derivatizing nanoparticles designed for renal clearance, it is imperative to use coatings that give the best compromise between, on one hand, the best protection and stabilization of the core and, on the other hand, the smallest size, or the material will not be filtered through the kidneys, and the branched PEG described in the present invention is eminently suited for this purpose. In particular would this be important in the context of nanoparticles containing materials foreign to the body, like transition metals, heavy metals, or lanthanides. These materials are of interest as diagnostics or therapeutics or both of the previous in combination. In particular, it is of interest to use heavy elements like gold, tantalum or tungsten incorporated in nanoparticles as contrast agents for CT (computed tomography) and they would benefit from being coated with a monolayer of the materials the general structures I or II. The efficacy of those materials is tightly correlated with the volume fraction of the heavy element in the nanoparticle and a coating with a monolayer of I or II will give materials with very good properties since the protective layer will be very thin while still achieving stabilization.

Linear PEG-derivatives with terminal alkylsilanoxy groups are suitable for surface modification of nanoparticles with a core of metal oxide. These siloxanes connect with hydroxyl groups present on the surface with the PEG tails oriented into the surrounding solution. Although, improved solubility and stability properties, there are still gaps in the PEG-coating exposing the core metal oxide due to the curvature of the particle surface and the folding of PEG-tails. Therefore an ideal coating molecule should contain one surface reactive group e.g. siloxane and two or more tails e.g. PEG producing a cone shaped structure. This type of branched PEG-siloxanes would lead to a denser PEG layer exposing less of the metal oxide core. In line with this reasoning, although not directly dealing with nanoparticles, Monfardini et al. (Bioconj. Chem. 1995, 6, 62-9) have shown that surface modification of proteins using branched PEG derivatives results in increase biocompability and better stability.

In particular would Iam with R¹═(R³)₃Si(CH₂)₃O— where R³ is as defined in table 1, Iae (R⁴═OH) with R¹═HO₂CCH₂O—, Is with R¹═HO₃S(CH₂)₃O—, lab with R¹═(HO)₂PO(CH₂)₃O—, Iq with R¹═HS(CH₂)₃O—, Iz with R¹═HSCH₂(HS)CHCH₂O— or Ian R¹═—O(CH₂)₂NHCO(CH₂)₄ CH(SH)CH₂CH₂SH be favourable for the stabilization of such particles. To our knowledge, no branched PEG-siloxanes are commercially available and have not been described in literature.

Luminescent nanoparticles, often called nanodots, based on various semiconductor materials like CdS, ZnS or InS, also need to be eliminated from the body through the kidneys and will benefit from being coated with a monolayer of I or II. In particular would I or II with Z selected from HS(CH₂)₃O—, HSCH(HS)(CH₂)₂O— or R¹═—O(CH₂)₂NHCO(CH₂)₄ CH(SH)CH₂CH₂SH be favourable for the stabilization of such particles.

In a fourth aspect of the present invention the molecules of general structures I or II are linked to a biomolecule like a protein, a peptide, a ribonucleic acid or a carbohydrate, forming structure III, or IV, where X is a residue of the group used for coupling and Y is a bond or a linker such as —NH(C═O)CH₂(OCH₂CH₂)ₙ— where n is selected from 1-50 and m is selected from 1-30 and A, B, and C are independently selected from —C₁-C₈ hydrocarbon, —(CH₂)₂N₃, —(CH₂)₂NH₂ and derivatives thereof such as, but not limited to amides, carbamates and ureas, CH₂COOH and derivatives thereof such as, but not limited to, esters, anhydrides, acid chlorides, amides, carbamates.

The purpose of this derivatization of the biomolecule is to enhance its properties with regard to solubility, reconstitutability after lyophilization, viscosity, stability to processing, stability in formulation, stability in-vivo, biological compatibility, immunogenicity, antigenicity, or pharmacokinetics or pharmacodynamics. The viscosity aspect is of considerable importance for products for injection and these more compact PEG derivatives give lower viscosities than the corresponding linear derivatives. In scheme 1 below is shown some methods to derivatize biomolecules in one or more positions.

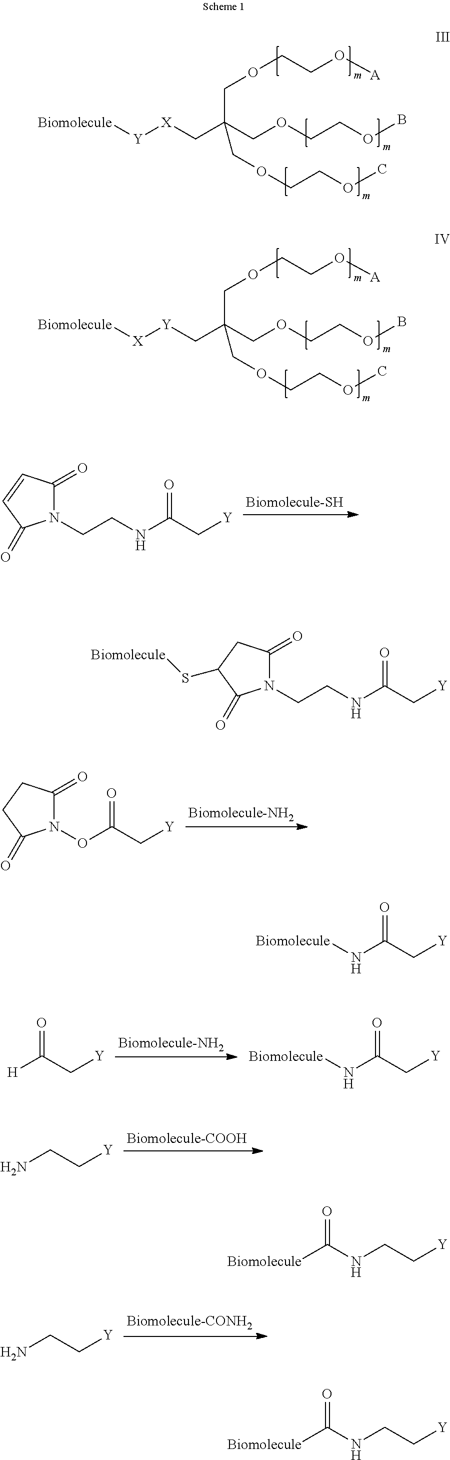

In a fifth aspect of the present invention the generic structures I or II is linked to a pharmaceutically active small molecule to enhance its properties with regard to solubility, viscosity, stability in formulation, stability in-vivo, biological compatibility, pharmacokinetics, or pharmacodynamics. Of particular importance is the effect to keep a drug out of the CNS since PEG derivatives lowers the ability of a drug to pass the blood brain barrier. The larger molecular size can also give slower clearance from the bloodstream and thus a more even drug concentration over time.

A non-limiting example where this concept has been used with polydisperse materials and where it would be beneficial to use the branched PEG derivative of the present invention can be found in WO2008112288 where opioids are directed to peripheral organs but not to the central nervous system.

For small molecules there is usually only one site of modification available so the branched structures of the present invention confer advantages since three PEG chains are introduced instead of one. The shorter chain length also lessens the likelihood of unwanted interference with the biological effect of the drug.

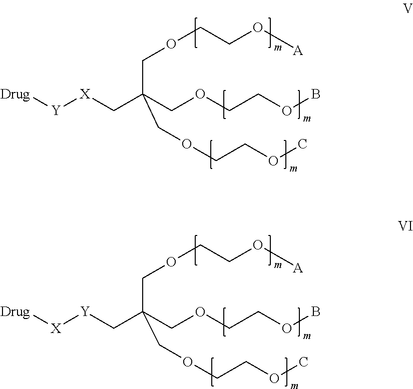

where X is a residue of the group used for coupling as outlined for the derivatization in scheme 1 and Y is a bond or a linker such as —NH(C═O)CH₂(OCH₂CH₂)ₙ— where n is selected from 1-50. A non-limiting example is structure Va where m=1-30 and A, B, and C are H or methyl. In one aspect m=1-10 and A,B, and C are methyl. In one aspect m=1-5 and A,B, and C are methyl.

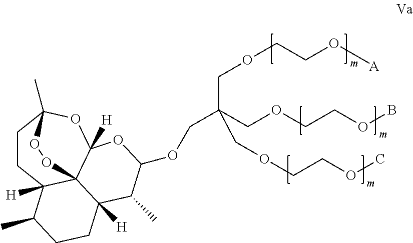

A number of trivial variations of the link betweeen the artemisinin moiety and the PEG can be envisioned. For example, a spacer group may be inserted and/or the link may contain an ester, amide or sulfonamide. It is also possible to substitute the hemiacetal oxygen with a sulfur, carbon or nitrogen atom.

In a sixth aspect of the present invention the molecules of general formula I or II is linked to a catalytically active molecule to form compounds VII or VIII, where X is a residue of the group used for coupling as outlined for the derivatization in scheme 1 and Y is a bond or a linker such as —NH(C═O)CH2(OCH₂CH₂)ₙ— where n is selected from 1-50. The catalyst would normally be a homogenous catalyst, to enhance its solubility in various solvents, particularly in water, improve stability in solid form or in solution, to modify solubility properties to facilitate removal from the product, or to enhance the activity of the catalyst. In particular this is of interest in the context of olefin metathesis catalysts, catalytic hydrogenation catalysts and cross-coupling catalysts.

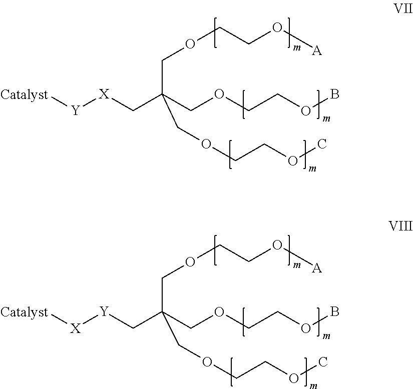

In Hong and Grubbs, J. Am. Chem. Soc. 2006, 128, 3508 is described a catalyst made water soluble by the derivatization with a polydisperse linear m-PEG through several synthetic steps and this material would be more tractable and hence cheaper to produce with a monodisperse PEG such as a PEG of general formula I or II instead.

Most homogenous catalysts have a phosphine as an integral part of the structure and it is often convenient to incorporate the solubilizing group I via the phosphine. There are many possibilities for this but if an aromatic phosphine is desired I or II can be linked via an ether oxygen. If an aliphatic phosphine is desired it is often possible to use I or II as a group linked directly to the phosphorous atom, introduced via alkylation.

It is also of importance that the more compact structure of the branched PEG derivatives of the present invention lowers the risk of interference with the catalytic process.

In a seventh aspect of the present invention, the general structures I or II is linked to a surface of a device. Of interest is a device for medical use and in particular when such a device is in contact with body fluids like a prostetic device or screw, a device through which body fluids are circulated and returned to the body, or an implant or an electrode implant. The surfaces of such devices may benefit from being coated with I or II to reduce the interaction with proteins of the body with the device. In particular this may reduce the formation of scar tissue around the device, reduce the formation of biofilms on the device, reduce the risk of infections around the device, reduce the risk of inflammation around the device, reduce the risk of corrosion of the device or reduce the risk of an immune response to the device.

The linking of the branched PEG derivative depends on the material of the device and there are a number of standard methods in the field. One is to use an oxy-silane like Iam in the presence of aqueous ammonia to attach the PEG derivative to the surface. Another is to use a phopsphonate like Iab, where the phosphonate has a high affinity for the surface.

In an eighth aspect of the present invention, the generic structures I or II is grafted to a polymeric structure (macromolecular framework), preferably of defined molecular weight or of low polydispersity. This has the advantage of creating materials of high but well defined molecular weight which may be of particular benefit when derivatizing proteins as mentioned above where relatively few attachment points are available. In scheme 2 is shown some representative structures according to this aspect.

In one aspect the fourth and the eighth aspect are combined. In one aspect the seventh and the eighth aspect are combined.

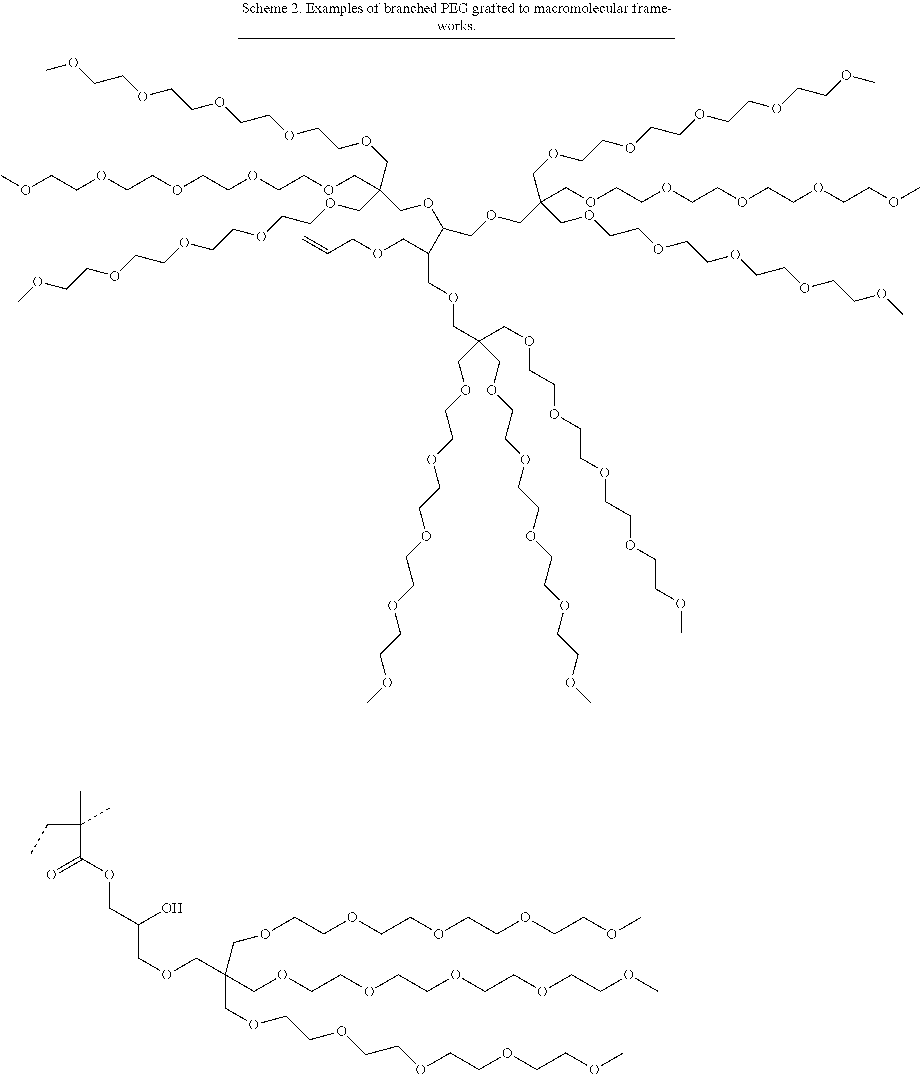

In a ninth aspect of the present invention general structures I and II are combined with a nonpolar moiety to yield amphiphilic molecules. There is an interest in using liposomes as vehicles for drug delivery and to suppress the immune response towards such products they are often derivatized with PEG on their surface. One aspect of the present invention is to use the molecules of general formula I or II linked to a lipid as part of the liposome. This has the advantage of giving a more uniform product and thus simplifying the regulatory process for approval. Structure IX with m selected from 1-30, for example 2-20, or, 3-12 or, 3-6 or 3-4. R⁶ and R⁷ are independently selected from C₈₋₂₅ hydrocarbon. A. B, and C are as previously defined. Other lamellar structures than liposomes may also be considered. Non-limiting examples are micells, inverted micells, vesicles and liquid crystals.

In one aspect of the invention m is 3-4.

Structure IXa is a generic structure of compounds suitable for the formation of liposomes according to the current invention. Structures IXb and 10 are non-limiting example suitable for the incorporation into liposomes or other

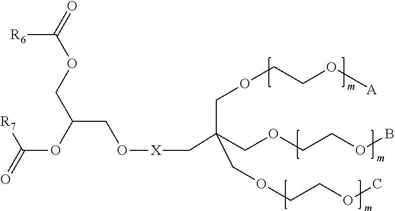

Structure IXa, X is a bond, a chain or a ring structure with 0-10 linear atoms chosen independently from C, O, N, P and S to form chemical compounds according to normal valency rules.

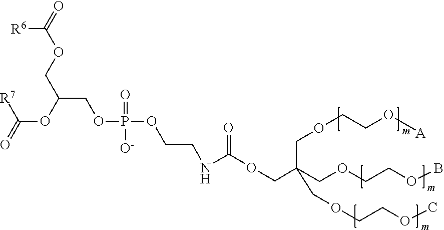

Structure IXb, m, A, B, and C are defined as for compounds I and II.

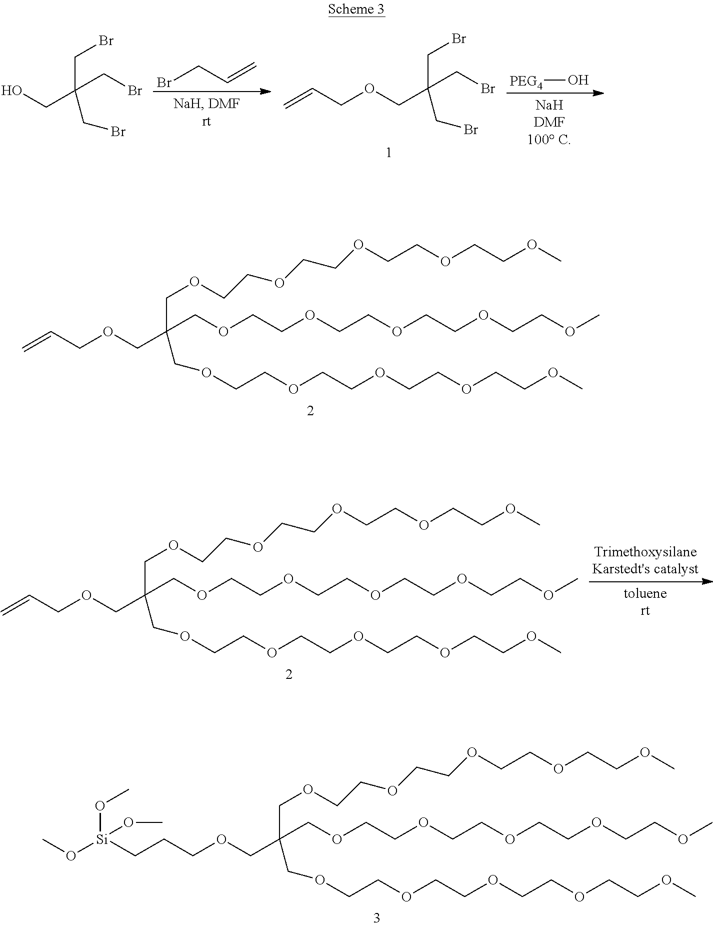

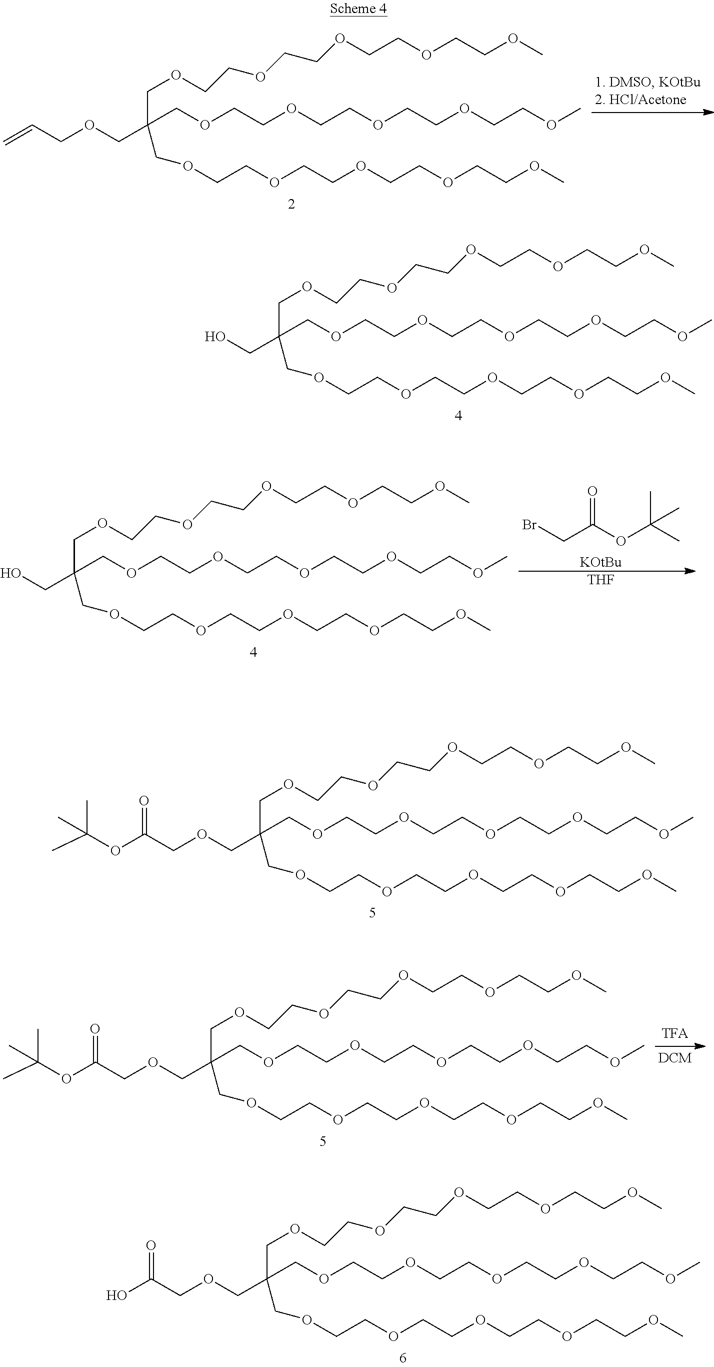

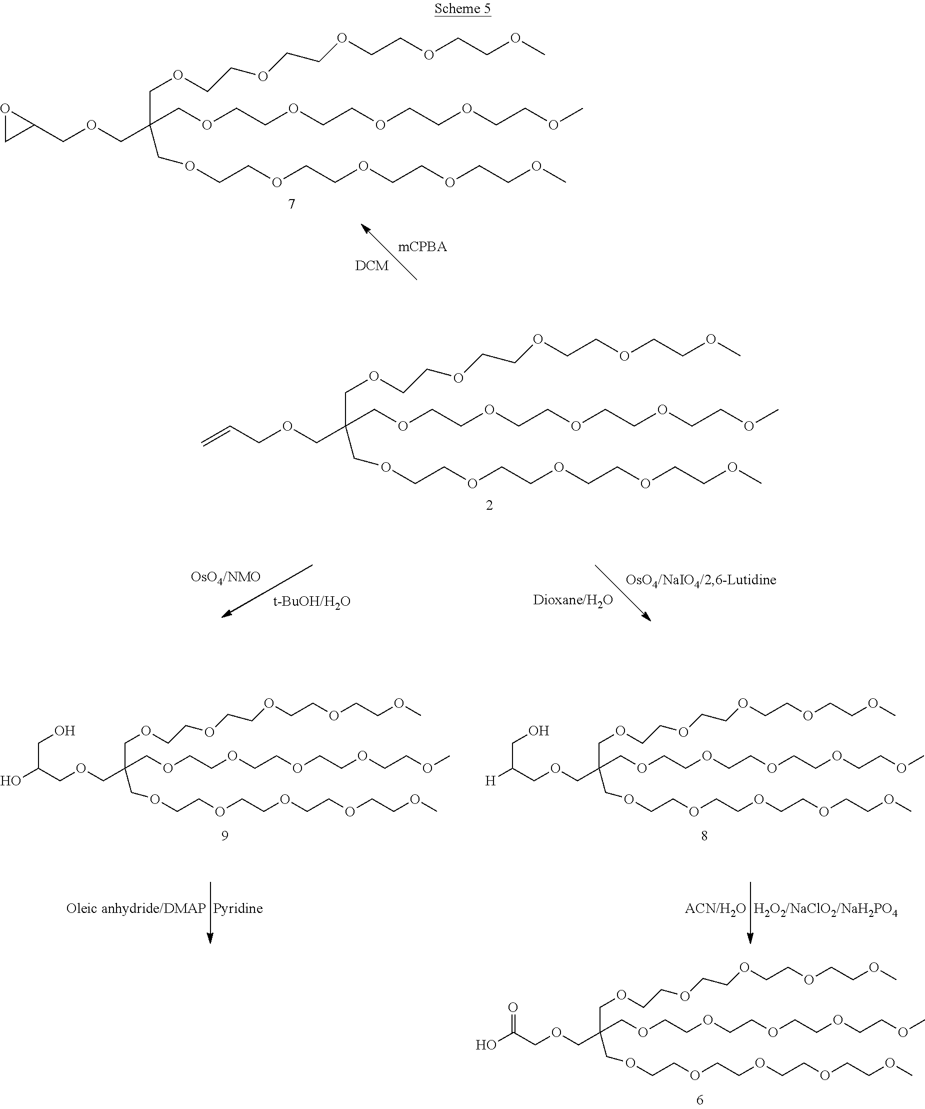

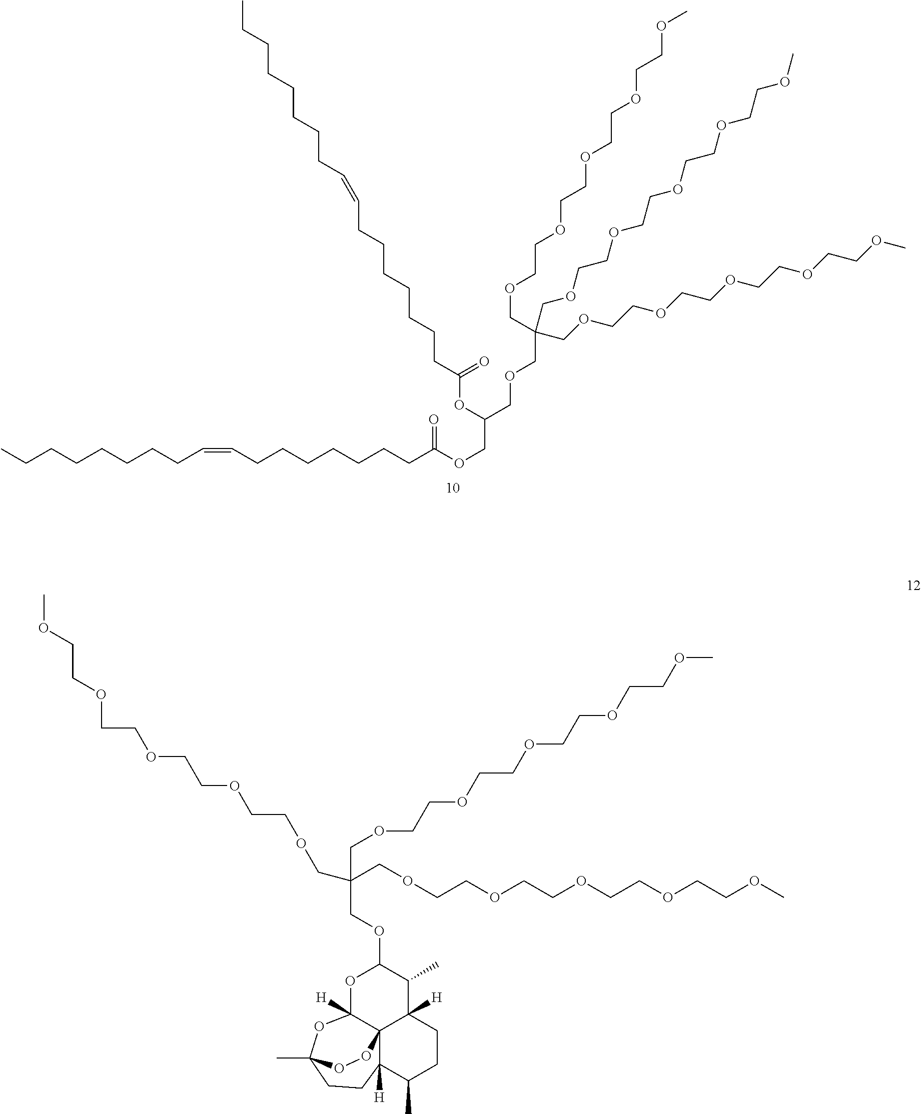 [SMILES: CCCCCCCC/C=C\CCCCCCCC(=O)OCC(COCC(COCCOCCOCCOCCOC)(COCCOCCOCCOCCOC)COCCOCCOCCOCCOC)OC(=O)CCCCCCC/C=C\CCCCCCCC ; COCCOCCOCCOCCOCC(COCCOCCOCCOCCOC)(COCCOCCOCCOCCOC)COC1O[C@H]2O[C@@]3(C)CC[C@H]4[C@H](C)CC[C@@H]([C@H]1C)[C@@]24OO3]

### BRIEF DESCRIPTION OF THE DRAWING

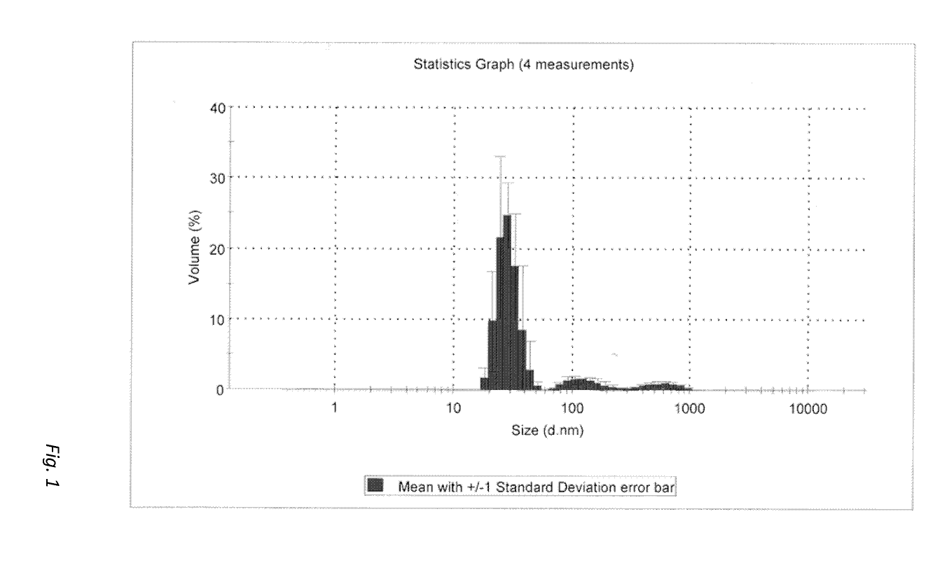

In the examples below reference is made to the drawing on which FIG. 1 illustrates DLS analysis of material from Example 11.

### EXAMPLES

### Example 1

### 3-(3-bromo-2,2-bis(bromomethyl)propoxy)prop-1-ene (1)

Sodium hydride (1.68 g, 42 mmol) was portion-wise added to 3-Bromo-2,2-bis(bromomethyl)propanol (9.75 g, 30 mmol) and allyl bromide (12.9 ml, 150 mmol) in dry and degassed (by vacuum) DMF (40 ml, 4 Å MS for 24 h) under nitrogen at 0° C. The temperature was then increased to room temperature (22° C.) and the reaction mixture was stirred for another 3 h. The reaction mixture was then carefully added to aqueous saturated NH₄Cl (50 ml). The H₂O-phase was then extracted with diethyl ether (2×50 ml) and the combined organic phases were washed with H₂O (5×50 ml) and the brine (50 ml). The organic phase was dried with Na₂SO₄ followed by filtration. The volatile materials were removed at reduced pressure to give a pale yellow oil (9.7 g). Column chromatography (heptane:EtOAc 9:1) gave 6.6 g (62%) of 1 as a clear oil. The product could also be isolated using distillation at reduced pressure, boiling point 85-87° C. at 0.05 mbar. ¹H-NMR (CDCl₃); 5.93 (m, 1H), 5.28 (m, 2H), 4.05 (d, 2H), 3.58 (s, 6H), 3.52 (s, 2H).

### Example 2

### 16-(allyloxymethyl)-16-2,5,8,11,14-pentaoxapentadecyl-2,5,8,11,14,18,21,24,27,30-decaoxahentriacontane (2)

Tetraethyleneglycol monomethyl ether (1.91 ml, 9 mmol) dissolved in dry and degassed DMF (3.5 ml, dried 24 h, 4 Å MS) was added carefully to sodium hydride (365 mg, 9 mmol) in dry and degassed DMF (15 ml, dried 24 h, 4 Å MS) under nitrogen at 0° C. using a syringe. The temperature was then raised to room temperature and the reaction mixture was stirred for another 30 min. Tribromide 1 (730 mg, 2.0 mmol) was then added and the temperature was raised to 100° C. After 14 h the reaction was completed (HPLC-ELSD-C18, 95:5 to 5:95 H₂O/ACN in 25 min, Rₜ product=19.5 min) the temperature was decreased to room temperature and the reaction mixture was carefully added to H₂O (150 ml) and the H₂O-phase was washed with diethyl ether (2×50 ml). Sodium chloride was then added to the H₂O-phase until saturation. The H₂O-phase was extracted with EtOAc (4×50 ml) and the combined organic phases were washed with brine (2×30 ml). Sodium sulfate and charcoal was added to the organic phase. The clear organic phase was filtered and the volatile material was removed at reduced pressure (8 mmHg, 40° C. then 0.1 mmHg (oil pump) and 40° C. to remove residual DMF). Column chromatography (EtOAc:MeOH 9:1) gave 1.05 g (70%) of 2. ¹H-NMR (CDCl₃); 5.90 (m, 1H), 5.20 (m, 2H), 3.94 (dt, 2H), 3.70-3.55 (m, 48H), 3.45 (s, 6H), 3.43 (s, 2H), 3.40 (s, 9H).

### Example 3

### 3,3-dimethoxy-9,9-di-2,5,8,11,14-pentaoxapentadecyl-2,7,11,14,17,20,23-heptaoxa-3-silatetracosane (3)

Platinum(0)-1,3-divinyl-1,1,3,3-tetramethylsiloxane (20 μl, 2% in xylene) was added to 2 (0.75 g, 1.0 mmol) and trimethoxysilane (255 μl, 2.0 mmol) in dry toluene (6 ml) under nitrogen at room temperature. The reaction mixture was shaken at room temperature for 24 h. Charcoal was then added and the reaction mixture was filtered after 2 minutes. The volatile material was removed at reduced pressure, which gave 830 mg (96%) of the title product containing 35% of 16-2,5,8,11,14-pentaoxapentadecyl-16-((prop-1-enyloxy)methyl)-2,5,8,11,14,18,21,24,27,-30-decaoxahentriacontane (HPLC-ELSD-C18, 95:5 to 5:95 H₂O/ACN in 25 min).

### Example 4

### 16,16-di-2,5,8,11,14-pentaoxapentadecyl-2,5,8,11,14-pentaoxaheptadecan-17-ol (4)

Potassium tert-butoxide (74 mg, 0.66 mmol) was added to 2 (500 mg, 0.66 mmol) in DMSO (3 ml). The reaction mixture was shaken at 100° C. for 15 min. HPLC analysis (HPLC-ELSD-C18, 95:5 to 5:95 H₂O/ACN in 25 min) indicated complete conversion to the product. Brine (20 ml) was added at room temperature and the aqueous phase was extracted with ethyl acetate (3×20 ml). The combined organic phases were washed with brine (3×20 ml) and the dried with sodium sulfate. Filtration and removal of volatile material at reduced pressure gave 16-2,5,8,11,14-pentaoxapentadecyl-16-((prop-1-enyloxy)methyl)-2,5,8,11,14,18,21,24,27,30-decaoxahentriacontane as a clear oil. HCl (0.1 M) was then added to the oil dissolved in acetone (4 ml) and the mixture was shaken at 55° C. for 30 min. The volatile material was then removed at reduced pressure, which gave 420 mg (89%) of 4 as a clear oil. ¹H-NMR (CDCl₃); 3.66-3.52 (m, 48H), 3.47 (s, 6H), 3.37 (s, 9H).

### Example 5

### tert-butyl 16,16-di-2,5,8,11,14-pentaoxapentadecyl-2,5,8,11,14,18-hexaoxaicosan-20-oate (5)

Potassium tert-Butoxide (32 mg, 0.28 mmol) was added to 4 (100 mg, 0.14 mmol) and tert-butyl-2-bromo acetate (105 mg, 0.54 mmol) in dry THF (3 ml). The reaction mixture was shaken for 30 min. Diethyl ether (10 ml and brine (5 ml) were added and the aqueous phase was extracted with ethyl acetate (3×20 ml). The combined organic phases were washed with brine and then dried with sodium sulfate. The volatile material was removed at reduced pressure and the crude product was purified by column chromatography (ethyl acetate/methanol 9:1), which gave 60 mg (52%) of 5. ¹H-NMR (CDCl₃); 3.91 (s, 2H), 3.66-3.52 (m, 48H), 3.51 (s, 2H), 3.45 (s, 6H), 3.37 (s, 9H), 1.46 (s, 9H).

### Example 6a

### 16,16-di-2,5,8,11,14-pentaoxapentadecyl-2,5,8,11,14,18-hexaoxaicosan-20-oic acid (6)

Trifluoroacetic acid (TFA, 0.5 ml) and dichloromethane (DCM, 0.5 ml) was added to 20 mg of 5. The mixture was shaken at room temperature for 1 h and the volatile materials was then removed at reduced pressure to give 18 mg of 6 as a yellow oil.

### Example 6b

### Alternative synthesis of 6,16-di-2,5,8,11,14-pentaoxapentadecyl-2,5,8,11,14,18-hexaoxaicosan-20-oic acid (6)

Hydrogen peroxide (145 mg, 4.26 mmol) dissolved in H₂O (9.5 ml) was added to mono sodium phosphate (588 mg, 3.87 mmol). This mixture was then trans-ferred to PEG aldehyde 8 (example 8, 2.20 g, 2.94 mmol) dissolved in acetonitrile (7 ml). The reaction mixture was cooled to 0° C. and sodium chlorite (898 mg, 7.94 mmol) in H₂O (9 ml) was added. The reaction mixture was stirred at room temperature for 4.5 h. Aqueous, ice cooled sodium metabisulfite (12 ml of 1M) was added and the mixture was stirred for another 25 min. The reaction mixture was then saturated with sodium chloride and the pH was raised to 7 by the addition of sodium hydroxide (1M). The mixture was extracted twice by EtOAc and then the pH of the aqueous phase was adjusted to 2 with HCl (6 M). The mixture was extracted with DCM (4×25 ml) and the combined organic phases were washed with brine and dried with sodium sulphate. Filtration followed by removal of the solvent at reduced pressure afforded 1.30 g of the product as an oil. HPLC analysis (HPLC-ELSD-C18, 90:10 to 10:90 H₂O/ACN in 20 min) displayed a single peak at 10.2 min.

### Example 7

### 16-(2-peroxypropoxymethyl)-16-2,5,8,11,14-pentaoxapentadecyl-2,5,8,11,14,18,21,24,27,30-decaoxahentriacontane (7)

To 3-chloroperoxy bensoic acid (247 mg, 1.0 mmol) was added to 6-(allyloxymethyl)-16-2,5,8,11,14-pentaoxapentadecyl-2,5,8,11,14,18,21,24,27,30-decaoxahentriacontane (example 2) (374 mg, 0.50 mmol) dissolved in DCM (10 ml) at room temperature. The reaction mixture was stirred for 72 h at room temperature and then washed with aqueous sodium bisulphite saturated with sodium chloride, aqueous saturated sodium bicarbonate, and finally, brine and then filtered. The solvent was removed at reduced pressure and toluene was added to the residue. The mixture was filtered and the solvent was removed from the filtrate at reduced pressure to give 320 mg of the product. HPLC analysis (HPLC-ELSD-C18, 90:10 to 10:90 H₂O/ACN in 20 min) displayed a single peak at 15 min.

### Example 8

### 16,16-di-2,5,8,11,14-pentaoxapentadecyl-2,5,8,11,14,18-hexaoxaicosan-20-al (8)

2,6 Lutidine (1.15 g, 10.7 mmol), osmiumtetraoxide (1.36 ml of 2% aqueous solution, 0.11 mmol) and sodiumperiodinate (4.58 g, 21.4 mmol) were consecutively added to 16-(allyloxymethyl)-16-2,5,8,11,14-pentaoxapentadecyl-2,5,8,11,14,18,21,24,27,30-decaoxahentriacontane (2) (4.0 g, 5.35 mmol) dissolved in dioxane/H₂O (3:1, 65 ml) at room temperature. The reaction mixture was stirred for 4.5 h and the volatile material was removed at reduced pressure. Brine (20 ml) was added to the residue and the aqueous phase was extracted with DCM (5×25 ml). The combined organic phases were washed twice with brine (20 ml) and then dried with sodium sulfate. Filtration and removal of the solvent at reduced pressure followed by column chromatography (DCM/methanol 95:5) of the crude product gave 2.65 g of the final product. HPLC analysis (HPLC-ELSD-C18, 90:10 to 10:90 H₂O/ACN in 20 min) displayed a single peak at 13.5 min. ¹H-NMR (CDCl₃); 9.70 (s, 1H), 4.01 (s, 2H), 3.7-3.4 (m, 56H), 3.37 (9H).

### Example 9

### 16-(2,3-dihydroxypropoxymethyl)-16-2,5,8,11,14-pentaoxapentadecyl-2,5,8,11,14,18,21,24,27,30-decaoxahentriacontane (9)

Osmium tetroxide (127 μl of a 2% aqueous solution, 0.01 mmol) followed by 4-methylmorpholine N-oxide (176 mg, 1,5 mmol) were added to 6-(allyloxymethyl)-16-2,5,8,11,14-pentaoxapentadecyl-2,5,8,11,14,18,21,24,27,30-decaoxahentriacontane (2) (747 mg, 1.0 mmol) in t-BuOH/H₂O 4:1 (5 ml). The reaction was stirred for 20 h at room temperature. Sodium bisulphate (100 mg) was added and the reaction mixture was stirred for another 10 min. The volatile materials were removed at reduced pressure and the residue was purified by column chromatography (DCM 100% to DCM/MeOH 9:1) which gave 600 mg of the product as a clear oil. HPLC analysis (HPLC-ELSD-C18, 90:10 to 10:90 H₂O/ACN in 20 min) displayed a single peak at 12 min. ¹H-NMR (CDCl₃); 3.66-3.52 (m, 48H), 3.47 (s, 6H), 3.37 (s, 9H).

### Example 10

### 16-(2,3-dioleyloxypropoxymethyl)-16-2,5,8,11,14-pentaoxapentadecyl-2,5,8,11,14,18,21,24,27,30-decaoxahentriacontane (10)

Oleic anhydride (492 mg, 0.9 mmol) dissolved in dry pyridine (0.5 ml, dried with 4 ÅMS) was added to PEG diol 9 (234 mg, 0.3 mmol) dissolved in dry pyridine (1 ml) at room temperature. N,N-dimethylaminopyridine (0.5 mg) was then added to the reaction mixture and the stirring was continued for 4 h at room temperature. More oleic anhydride (246 mg, 0.45 mmol) was then added and the reaction mixture was stirred for another 20 h. The volatiles were removed at reduced pressure and the residue was purified by column chromatography (DCM 100% to DCM/MeOH 95:5) which afforded 360 mg of the product as a clear oil. ¹H-NMR (CDCl₃); 5.36 (m, 4H), 5.20 (m, 1H), 4.33 (dd, 1H, J=12.0 and 3.2 Hz), 4.14 (dd, 1H, J=12 and 6.4 Hz), 3.70-3.50 (m, 50H), 3.44 (s, 2H), 3.42 (6H), 3.40 (9H), 2.31 (m, 4H), 2.03 (m, 8H), 1.62 (m, 4H), 1.30 (m, 44H), 0.90 (t, 6H, J=7.2 Hz).

### Example 11

### Liposomes of 10

PEG-glyceryldioleate 10 (20 mg) was dissolved in H₂O (1 ml). The mixture was shaken for 30 min at 37° C. and 30 min at room temperature. The clear solution was filtered (0.2 um syringe filter) and the size of the generated nanostructure was determined by DLS (Malvern Zetasizer) to have a typical hydrodynamic diameter of 30 nm (volume average).

The results of DLS analysis of material from example 11 are shown in FIG. 1.

### Example 12

### Dihydroartemisinin-PEG conjugate 12

Bortrifluoride etherate (104 μl, 1.0 mmol) was added to 16-(hydroxymethyl)-16-2,5,8,11,14-pentaoxapentadecyl-2,5,8,11,14,18,21,24,27,30-decaoxahentriacontane (Ia with m=4; A, B, C=Me)(707 mg, 1.0 mmol) and dihydroartemisinin (426 mg, 1.5 mmol) in dry diethyl ether (20 ml) at room temperature. After 4 h, more bortrifluoride etherate (31 μl, 0.25 mmol) was added and the reaction mixture was stirred for another 20 h. Dilute aqueous sodium bicarbonate (2%) was added and the reaction mixture was stirred for another 30 min. The reaction mixture was extracted with EtOAc (3×30 ml) followed by DCM (2×25 ml). The combined organic phases were dried with sodium sulphate. Filtration and removal of the solvent at reduced pressure followed by column chromatography (EtOAc/heptane 1:1 then DCM/MeOH 9:1) of the crude product gave 780 mg of the product. HPLC analysis (HPLC-ELSD-C18, 90:10 to 5:95 H₂O/ACN in 20 min, then 5 min 5:95 H₂O/ACN) displayed the product peak at 23.5 min. MS (ESP+) [M+Na⁺]: 995.6.

1. A branched polyethyleneglycol (PEG) derivative consisting of a molecule with a quaternary carbon connected to three PEG chains, wherein all three PEG chains are of equal length and each comprises 1-30-OCH₂CH₂— units, and one group with at least one carbon atom, wherein said at least one carbon atom is attached to the quaternary carbon.

2. A branched PEG derivative according to claim 1, having the general formula I or II 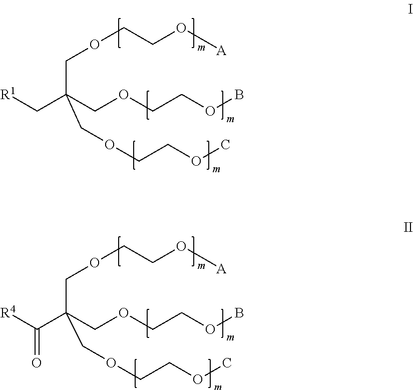
wherein:
R¹ is selected from the group consisting of —OH, —OSO₂CH₃, —OSO₂PhCH₃, —OCH₂CH═CH₂, —OBenzyl, -halogen, —NH₂, —NHCO₂R², —NHCONHR², —NHCON(R²)₂, —NCO, —NHCO(CH₂)₄ CH(SH)CH₂CH₂SH, —NHCOCH₂SH, —SH, —SR², —SO₃H, —O(CH₂)₃SH, —OCH₂CH(SH)CH₂SH, —OCO(CH₂)₄ CH(SH)CH₂CH₂SH, —PO₃H₂, —PO3(R²)₂, —OCH₂COOH, —OCH₂COR⁴, —O(CH₂)₂NH₂, —O(CH₂)₂NHR², —O(CH₂)₂N(R²)₂, —O(CH₂)₂NHCO₂R², —O(CH₂)₂NHCONHR², —O(CH₂)₂NHCON(R²)₂, —O(CH₂)₂NCO, —O(CH₂)₃Si(R³)₃, —O(CH₂)₂NHCO(CH₂)₄ and CH(SH)CH₂CH₂SH;
A, B and C are independently selected from the group consisting of —C₁-C₈ hydrocarbon, —(CH₂)₂N₃, —(CH₂)₂NR⁵₂ and —CH₂COOR⁴;
R² is selected from the group consisting of C₁-C₈ hydrocarbons;
R³ is selected from the group consisting of C₁₋₈ alkoxy groups, aryloxy group, a halogen, a di-C₁₋₈-alkylamino group, a nitrogen containing heterocycle or an acyloxy group;
R⁴ is selected from the group consisting of H, OH, OR², NHR², N(R²)₂, halogen, N-hydroxysuccinimidyl (NHS ester) and perfluorophenolate; and
R⁵ is independently selected from the group consisting of —H, —C₁-C₈ hydrocarbon, —(C═O)NH₂, —(C═O)—OCH₃, —OCH₂CH₃ and m is selected from 1-30.

3. A branched PEG derivative according to claim 1, wherein m is 3-20.

4. A branched PEG derivative according to claim 1, wherein m is 3-10.

5. A branched PEG derivative according to claim 1, wherein m is 3-5.

6. A branched PEG derivative according to claim 2, wherein A, B and C all are the same.

7. A branched PEG derivative according to claim 6, wherein A, B and C all are —CH₃.

8. A branched PEG derivative according to claim 2, wherein said compound has formula I and wherein R¹ is O(CH₂)₃Si(R³)₃, wherein R³ is selected from the group consisting of C₁₋₈ alkoxy, acyloxy, dialkylamino and aryloxy.

9. A branched PEG derivative according to claim 2, wherein said compound has formula I and wherein R¹ is CH₂NH₂ or OCH₂COOH.

10. A branched PEG derivative according to claim 1 having one of the following two general formulas: 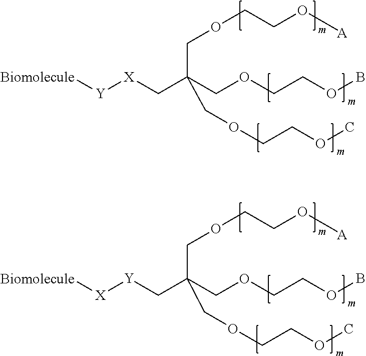
wherein:
“Biomolecule” denotes a peptide or an antibody fragment or a ribonucleic acid or a carbohydrate;
X is a residue of the functionality used for coupling;
Y is a spacer group with the structure —(OCH2CH2)ₙO—, wherein n is 1-50;
m is 2-30;
A, B and C are independently selected from the group consisting of —C₁-C₈ hydrocarbon, —(CH₂)₂N₃, —(CH₂)₂NR⁵₂ and —CH₂COOR⁴;
R² is selected from the group consisting of C₁-C₈ hydrocarbons;
R³ is selected from the group consisting of from C₁₋₈ alkoxy groups, aryloxy group, halogens, di-C₁₋₈-alkylamino groups, nitrogen containing heterocycles or acyloxy groups;
R⁴ is selected from the group consisting of H, OH, OR², NHR², N(R²)₂, halogen, N-hydroxysuccinimidyl (NHS ester) and perfluorophenolate;
and R⁵ is independently selected from the group consisting of —H, —C₁-C₈ hydrocarbon, —(C═O)NH₂, —(C═O)—OCH₃, and —OCH₂CH₃.

11. A branched PEG derivative according to claim 1 having one of the 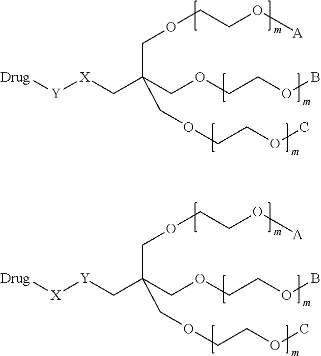
following two general formulas:
wherein:
“Drug” denotes a pharmaceutically active drug of molecular weight less than 1000 g/mol;
X is a residue of the functionality used for coupling;
Y is a spacer group with the structure —(OCH2CH2)ₙO—, wherein n is 1-50,
m is 2-30;
A, B and C are independently selected from the group consisting of —C₁-C₈ hydrocarbon, —(CH₂)₂N₃, —(CH₂)₂NR⁵₂ and —CH₂COOR⁴;
R² is selected from the group consisting of C₁-C₈ hydrocarbons;
R³ is selected from the group consisting of C₁₋₈ alkoxy groups, aryloxy groups, halogens, di-C₁₋₃-alkylamino groups, nitrogen containing heterocycles and acyloxy groups;
R⁴ is selected from the group consisting of H, OH, OR², NHR², N(R²)₂, halogen, N-hydroxysuccinimidyl (NHS ester) and perfluorophenolate;
and R⁵ is independently selected from the group consisting of —H, —C₁-C₈ hydrocarbon, —(C═O)NH₂, and —(C═O)—OCH₃.

12. A branched PEG derivative according to claim 1, having the general formula Va 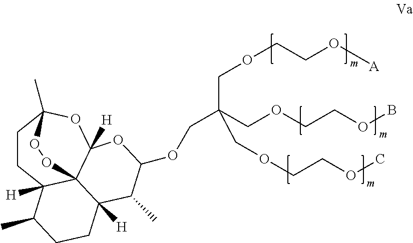
wherein m=1-30 and A, B, and C independently are selected from the group consisting of H and methyl.

13. A branched PEG derivative according to claim 1, having the general formula IXa 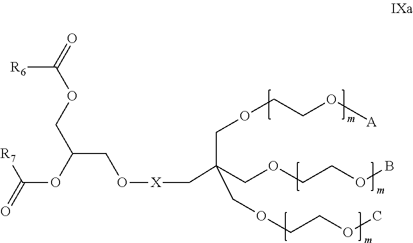
wherein:
R⁶ and R⁷ are independently selected from the group consisting of C₈-C₂₅ hydrocarbons;
m is selected from 2-30;
A, B and C are independently selected from the group consisting of —C₁-C₈ hydrocarbon, —(CH₂)₂N₃, —(CH₂)₂NR⁵₂ and —CH₂COOR⁴;
R² is selected from C₁-C₈ hydrocarbon;
R³ is selected from C₁₋₈ alkoxy group, aryloxy group, a halogen, a di-C₁₋₈-alkylamino group, a nitrogen containing heterocycle or an acyloxy group;
R⁴ is selected from H, OH, OR², NHR², N(R²)₂, halogen, N-hydroxysuccinimidyl (NHS ester) and perfluorophenolate;
R⁵ is independently selected from —H, —C₁-C₈ hydrocarbon, —(C═O)NH₂, —(C═O)—OCH₃, or —OCH₂CH₃;
X is a bond, a chain or a ring structure with 0-10 linear atoms chosen independently from C, O, N, P and S to form chemical compounds according to normal valency rules;

14. A branched PEG derivatives according to claim 1 where the quaternary central carbon originates from 3-halo-2,2-bis(halomethyl)propanol.

15. A branched PEG derivative according to claim 14, wherein the 3-halo-2,2-bis(halomethyl)propanol is 3-bromo-2,2-bis(bromomethyl)propanol.

16. A composition comprising branched PEG derivatives according to claim 1.

17. A composition comprising nanoparticles with a surface coating comprising branched PEG derivatives according to claim 1.

18. A composition comprising branched PEG derivatives according to claim 13, wherein said composition comprises liposomes comprising said branched PEG derivatives.

19. A composition according to claim 16, wherein a major part of the composition consists of branched PEG derivatives.

20. A composition according to claim 16, wherein the branched PEG derivatives are of defined molecular weight or monodisperse so that the purity of the product is more than 50%.

21. A composition according to claim 20, wherein the purity is more than 80%.

22. A composition according to claim 20, wherein the purity is more than 90%.

23. A composition according to claim 20, wherein the purity is more than 95%.

24. A composition according to claim 20, wherein the purity is more than 99%.

25. A method for producing branched PEG derivatives according to claim 1, wherein a solution of (3-halo-2,2-bis(halomethyl)propanol) in an aprotic solvent is contacted with A-(OCH₂CH₂)ₙO⁻; or a base and A-(OCH₂CH₂)ₙOH wherein A is selected from C₁₋₈ hydrocarbon, benzyl, N₃CH₂, —(CH₂)₂NH₂, or CH₂COOR wherein R is a C₁₋₈ hydrocarbon, and wherein said solution is heated at temperature of 30-150° C. for a duration from 30 minutes to 30 hours.

26. A method according to claim 25, wherein said solution is heated at a temperature of 70-120° C. for a duration from 30 minutes to 30 hours.

27. A method according to claim 25, wherein said solution is heated at a temperature of 90-110° C. for a duration from 30 minutes to 30 hours.

28. Use of a branched PEG derivative according to claim 1 for structural modification of a biomolecule selected from the group consisting of proteins, peptides, antibodies, antibody fragments, carbohydrates, and nucleic acids or small molecule drugs.

29. Use of a branched PEG derivative according to claim 1 in a nanoparticle.

30. Use of a branched PEG derivative according to claim 1 in a homogeneous catalyst.

31. Use of a branched PEG derivative according to claim 1 in a medical device.

32. A medical device, or a prostetic device or a screw, or a device through which body fluids are circulated and returned to the body, or an implant or an electrode implant with a surface coating comprising a branched PEG derivative according to claim 1.
